(19) United States
(12) Patent Application Publication
(10) Pub. No.: US 2008/0097199 A1
(43) Pub. Date: Apr. 24, 2008

(54) Tissue Marking Devices and Systems

(76) Inventor: David Mullen, Old Greenwich, CT (US)

Correspondence Address: MCDERMOTT WILL & EMERY LLP, 600 13TH STREET, N.W., WASHINGTON, DC, 20005-3096, US

(21) Appl. No.: 11/660,205

(22) PCT Filed: Nov. 12, 2004
(86) PCT No.: PCT/US04/37605

§ 371 Date: Feb. 15, 2007

Related U.S. Application Data
(60) Provisional application No. 60/602,876, filed on Aug. 20, 2004.
(60) Provisional application No. 60/614,990, filed on Oct. 4, 2004.

(51) Int. Cl. A61B 10/00

(52) U.S. Cl. 600/431

(57) ABSTRACT

A marker for marking a site within the body of a mammalian patient is positioned within the tissue of a patient. The marker may be placed in the first instance by a needle or the like or placed where a tissue sample has been removed. The marker has a plurality of loops each at various angles to the other such that when positioned within the patient, one of the loops is positioned orthogonal to a magnetic field of a metal detector. Various shapes of markers may be used, including electron orbital shapes, chains of loops or barbells. Barbs or other anchoring elements may be used to stabilize the marker's position. Normal delivery techniques as needles, catheters or cannulas may readily position the marker within the patent. By having the marker so designed and positioned, at least one of the closed loops is detectable by a metal detection beam of a metal detection device.

### FIELD OF THE INVENTION

The present invention relates to implantable and readily detectable medical devices and systems useful for marking a tissue lesion in a subject for later surgical removal.

### BACKGROUND

The need for accurate preoperative image guided localization of nonpalpable breast lesions has been well described, and the frequency of use for this technique is increasing. Not only mammographically detected lesions require localization, but also lesions that may be found by any other imaging technique such as ultrasound, MRI, nuclear medicine or other technologies not yet described. Such localizations generally require the positioning of a temporary marker, most frequently constructed of a metal anchor on the end of a wire inserted through a needle that has been accurately positioned by image guidance prior to the release of the marker. See, Frank H. A., Hall F. M., Steer M. L., Preoperative Localization of Nonpalpable Breast Lesions Demonstrated by Mammography; New England Journal of Medicine 1976; 296:259-260. In addition, the implantation of a small metal “clip” marker following large core biopsy under image guidance may be used when the visualized target has been substantially removed during the diagnostic procedure (thus compromising future successful localization). See, Burbank F. et al., “Tissue Marking Clip for Stereotactic Breast Biopsy: Initial Placement Accuracy, Long-term Stability, and Usefulness as a Guide for Wire Localization”; Radiology 1997; 205:407-415.

The need for such localization is best understood in the breast but will be of growing importance in other organ systems. The explosive growth of diagnostic imaging has increased the frequency of detection of small lesions throughout the body that cannot be seen or felt by a surgeon charged with the task of removing such a target.

Guidance for radiation therapy or other emerging ablation techniques using thermal, laser, radiofrequency or other methods of local energy or drug deposition to kill cells is also needed.

Many metal devices to accomplish breast marking or localization have been devised (e.g., U.S. Pat. Nos. 4,799,495; 5,011,473; 5,057,085; 5,083,570; 5,127,916; 5,158,084; 5,221,269; 5,234,426; 5,409,004; 5,556,410; 6,053,925; and 6,544,269). These devices all have the significant limitation of requiring the image guided localization procedure immediately before the surgery. Because the anchoring device is connected to a wire that protrudes through the skin, it must be promptly removed. Even those devices now approved for implantation into the breast (following the special case of a small lesion which has been substantially removed during image guided large core needle biopsy) must be re-localized with a second temporary device on the day of definitive surgery. The need for immediate preoperative localization creates logistical bottlenecks for radiology departments and operating room throughput, as well as additional procedures for patients who have already had a device implanted.

Thus, a device that could be implanted by a radiologist at one time and then independently removed by a surgeon at another time on the day of the needed surgical procedure with no further patient preparation is desirable. Such a device should serve as a marker placed when the need for surgical guidance is already specifically known. Additionally, such a device should be able to mark the site of a large core percutaneous biopsy when the need for surgery at some future point is considered likely. Although some have attempted solutions to these problems, a simple and cost effective approach has not yet been found.

Before the era of diagnostic breast imaging, only palpable lesions were detectable. Palpable lesions can be biopsied by a surgeon without any form of marking or guidance other than physical examination of the breast before and during the surgical procedure. Lesions that are detectable only by imaging, however, are best biopsied after marking. Although this is now done following wire localization, some have suggested the use of markers that may render a previously nonpalpable lesion palpable, thus providing the surgeon with a familiar method of tactile guidance. See, Debbas, Apparatus for Locating a Breast Mass, U.S. Pat. No. 5,662,674; Fulton et al. Biopsy Localization Method and Device, U.S. Pat. No. 6,730,042; and Fulton et al. Target Tissue Localization Device and Method, U.S. Pat. No. 6,409,742. These proposed devices all have one significant drawback in common: they are large and may be expected to be uncomfortable for patients. This problem may be further compounded when these may need to remain in position for some length of time.

Some lesions may be located by ultrasound. In addition, methods to use ultrasound to identify the site of a previous needle biopsy have been described. The hematoma that sometimes occurs following a biopsy procedure may be recognized sonographically until it is reabsorbed. Some have considered the possibility of intentionally creating such a hematoma by injecting a patient's own blood into a biopsy cavity. See, Klimberg et al., Method for Detecting and Excising Nonpalpable Lesions, U.S. Pat. No. 6,714,808. Devices have been designed to implant collagen or other bioabsorbable materials following a large core biopsy. See, Burbank et al., Methods and Chemical Preparations for Time-Limited Marking of Biopsy Sites, U.S. Pat. No. 6,427,081; Montegrande, Ultrasound Imaging Marker and Method of Use, U.S. Pat. No. 6,544,185; Fisher, Bioabsorbable Markers for Use in Biopsy Procedures, U.S. Pat. No. 6,350,244. Such materials may temporarily increase the conspicuity of the marked region. Although these techniques may work in certain circumstances for preoperative wire localization, they have significant limitations for use during operations. Generally, lesions, as well as markers, are subtly revealed. These lesions may be further obscured by blood or air as a surgical dissection proceeds. Ultrasound equipment is expensive and requires substantial training to use effectively. Significant operator dependency is a well recognized feature of sonographic examination and the use of skilled technologists trained in this technique is common. Such technologists are in short supply and are usually more effectively utilized within a radiology department than in an operating room. Although some have proposed that surgeons use ultrasound themselves in the operating room, the impediments described above have made this practice uncommon. In fact, the vast majority of ultrasound localization procedures are still performed with a wire device by a radiologist on the day of surgery.

The intraoperative use of small handheld radiation detection probes to guide surgeons to “sentinel” lymph nodes has been well described and is growing in clinical use. Some have experimented with the use of these probes to guide surgeons to a desired target within the breast by injecting radioactive drugs, or placing radioactive metal “seeds” directly into the target. See, Tanis, P. J. et al., Single Intralesional Tracer Dose of Radio-Guided Excision of Clinically Occult Breast Cancer and Sentinel Node, Annals of Surgical Oncology, 2001; 8(10):850-855. A randomized controlled comparison of wire versus seed localization revealed that seed localization with probe detection was easily learned by radiologists and surgeons. See, Gray, R. J. et al., Randomized Prospective Evaluation of a Novel Technique for Biopsy or Lumpectomy of Nonpalpable Breast Lesions: Radioactive Seed Versus Wire Localization, Annals of Surgical Oncology, 2001; 8(9):711-715. Probe based guidance for the lesions marked by radioactive seeds reduced the rate of inadequate tumor margins by more than 50% when compared with wire localization, with no other differences between the two techniques. This result suggests there may be great benefit of probe-based detection.

Nevertheless, nuclear techniques are cumbersome not only because of the patient preparation needed, but also the nature of radioactive products. The expense, licensure requirements and handling regulations for such products are significant. The physical decay characteristics of radioactive materials are also problematic. If short half-life materials are used, the surgery must occur promptly before decay, while if long half-life materials are used, the device must be removed promptly to limit radiation to the nearby normal tissues. Radioactive decay may similarly limit shelf life or complicate long-term storage. These limitations will of necessity impact the practicality of these techniques.

Devices that emit energy other than by radioactive decay have been described. These may serve as a “homing beacon” during an operation. Markers that emit light to enhance detection by allowing the surgeon to visually identify the localized region without following the course of insertion have been devised. See, Hussman, Method for Localizing a Lesion using an Optical Fiber, U.S. Pat. No. 5,954,655. Similarly, radio frequency transmitters that may be detected by probes have been the subject of prior patent application, Field S. E., Position Sensing System and Method for Using the Same, U.S. Pat. No. 6,006,750. Although these may enhance detectabilty of the marked region, they suffer from the need for power from a source of energy. Thus, such devices may require a wire or fiber connected to a power source outside of the body. This, like traditional wire localization, precludes decoupling of the insertion with the surgery.

Although “wireless” markers have been envisioned for breast marking, the actual physical structure of such a marker has not been well defined. See Krag D. N., System and method for bracketing and removing tissue, U.S. Pat. No. 6,698,433. A wireless marker for radiation therapy guidance has been described; see Mate, T. P., Dimmer, S. C., U.S. patent application 09-877498, but this has no mechanism to provide for positional stability in loose soft tissues such as breast or lung and thus may be prone to movement, particularly when inserted in a cavity following large core biopsy.

Techniques for the intraoperative localization of brain lesions based on interactive computerized programs that relate external landmarks or fiducial markers to the internal contents of the cranium are now available. These allow a previously obtained imaging study of the brain to provide a surgeon with the capacity to point with a probe at the patient's head while a virtual representation of the relationship of the target to the probe is created in a dynamic display. These techniques have been extended from the brain to the face and sinuses. Although some have imagined that such techniques may be adapted for use in the breast or elsewhere in the body, Vilsmeier et al., Method for the Localization of Targeted Treatment Areas in Soft Body Parts, U.S. Pat. No. 6,424,856; Front et al., Method and System for Guiding a Diagnostic or Therapeutic Instrument Towards a Target Treatment Inside the Patient's Body, U.S. Pat. No. 6,567,687; Spigelman et al., Systems and Methods for Targeting a Lesion, U.S. Pat. No. 6,731,966; and Kalfas et al., Frameless Stereotaxy System for Indicating the Position and Axis of a Surgical Probe, U.S. Pat. No. 5,776,064, there are reasons to believe that this may be impractical in some organs. Unlike the brain and facial structures, which are contained within the rigid skull and facial bones, organs like the breast are soft and changeable in shape. No rigid external landmarks exist for points of reference. Other organs may have rigid external reference points but change shape dramatically during surgery, such as the lung, which is purposefully deflated during an operation.

A technique to mark tissue with sterile charcoal at any time prior to breast surgery has been described, Svane G. A., A Stereotaxic Technique for Preoperative Marking of Non-palpable Breast Lesions, Acta Radiol Diagn (Stockh) 1983; 24:145-151. This technique has been the subject of additional published studies; see Mullen, D. J. et al., The Use of Carbon Marking after Stereotactic Large-core-needle Breast Biopsy. Radiology 2001; 218:255-260.; Canavese, G., et al., Preoperative Localization of Nonpalpable Lesions in Breast cancer by Charcoal Suspension, European Journal of Surgical Oncology; 1995; 21: 47-49.; Delporte, P., et al. Preoperative Localization of Asymptomatic Breast Lesions by the Technique of Stereotaxic Tattooing and Use of a Wire, (French) Chir Paris 1994 131; 12: 549-553; Langlois, S., et al., Carbon Localisation of Impalpable Mammographic Abnormalities, Australas Radiol 1991; 35: 237-241; and Rose, A., et al., Carbon Localisation of Impalpable Breast Lesions, The Breast 2003 12: 264-269. This method has the benefit of completely decoupling the time of localization from the time of surgery. Although this technique has repeatedly been reported as safe, effective and inexpensive, its adoption has remained limited.

The suggested use of metal detectors for surgical guidance has been occasionally reported since the introduction of this concept by Alexander Graham Bell, who used his invention during attempts to retrieve the bullet lodged within President Garfield following his shooting in 1881. See, Brown R. J., Alexander Graham Bell and the Garfield Assassination, http://www.historybuff.com/library/refgaffield.html. Although metal detection by treasure hunters and at security checkpoints is now commonplace, the modern medical use of this technique has remained limited. By way of example, the localization of metal foreign bodies such as accidental metal ingestions; Arena, L. et al., Use of a Metal Detector to Identify Ingested Metallic Foreign Bodies, American Journal of Roentgenology 1990; 115:803-804; missile wounds; Veselko, M. et al. Intraoperative Localization of Retained Metallic Fragments in Missile Wounds, Journal of Trauma, 2000; 49:1052-1058; broken surgical instruments; Moore, N. J. et al., The Use of a Metal Detector for Localisation of a Metallic Foreign Body in the Floor of the Mouth, British Journal of Oral and Maxillofacial Surgery (1993) 31:191-192; and orthopedic screws; Trobec R. et al., Metal Implant Localizers: Frontiers and Diagnostic Feasibility, Journal of Medical Engineering & Technology 1996 (May/June); 20(3):134-140.

While the above literature reports the use of metal detection as safe and effective, none of the patents, publication or literature described above show an implantable medical device for marking a site within the body of a patient from which a tissue sample is to be taken or has already been removed that is capable of being detected by a metal detection beam of a metal detector. Further, none of the literature suggests the use of such a marker and detector to provide intentional directional assistance to a surgeon by deliberately placing a marking device that can be subsequently accurately detected and reliably removed.

### SUMMARY OF THE INVENTION

According to the invention, an implantable medical device or marker is described for marking a site within the body of a patient from which a tissue sample is to be taken or has already been removed, where the medical device is capable of being detectable by a metal detection beam of a metal detection device. The marker has one or more loops and/or positioning elements thereon such that when positioned within a patient, at least one of loops would be orthogonal to a metal detection beam for a given directional source of the beam.

In another aspect of the invention, the medical device has a plurality of loops wherein the device is disposed to present at least one of the loops being substantially parallel to the metal detection beam for a-given directional source of the beam.

Another aspect of the invention relates to construction of a metallic composition that is most suitable for detection by a metal detection beam of a metal detector.

Another aspect of the invention relates to devices made of a metallic material such that the devices may be delivered to a desired location in the body in compressed form and then expanded upon deployment.

Another aspect of the invention relates to a device that, by nature of its shape, will maximize detectability regardless of the detection device employed.

Another aspect of the invention relates to delivery systems adapted for use with multiple differing needle types and biopsy instruments.

Additional advantages of the present invention will become readily apparent to those skilled in this art from the following detailed description, wherein only a few exemplary embodiments—of the invention are shown and described, simply by way of illustration of the best mode contemplated of carrying out the invention. As will be realized, the invention is capable of other and different embodiments, and its several details are capable of modifications in various obvious respects, all without departing from the invention. The present invention may be practiced without some or all of these specific details. In other instances, well known process operations have not been described in detail, in order not to unnecessarily obscure the present invention.

Accordingly, the drawings and description are to be regarded as illustrative in nature, and not as restrictive. The invention itself, together with further objects and attendant advantages, will be understood by reference to the following detailed description, taken in conjunction with the accompanying drawings.

### BRIEF DESCRIPTION OF THE DRAWINGS

FIG. 1(a) schematically illustrates a metal detection probe approaching a patient's breast.

FIG. 1(b) is an enlargement of the area of FIG. 1(a) shown in dotted lines, illustrating the probe inserted through a surgical dissection incision into the patient's breast and approaching a marker according to the present invention.

FIG. 2(a) shows the shape and direction of a magnetic field created by current flowing in a loop (as in a metal detector).

FIG. 2(b) shows the induction of current in a closed conducting loop by a changing magnetic field.

FIG. 2(c) shows the magnetization of a ferromagnetic object in a magnetic field.

FIG. 2(d) shows the combined effects of the current induction and magnetization.

FIG. 2(e) shows a marker according to the present invention within a magnetic field.

FIGS. 3(a) to 3(f) show markers of the present invention having shapes patterned from electron orbitals.

FIGS. 4(a)-1 to 4(e) show markers of the present invention having shapes patterned from chain-like configurations of loops.

FIGS. 5(a) to 5(e) show markers of the present invention having shapes patterned from barbell shapes.

FIG. 6 shows a flexible cannula having a marker positioned within a Mammotome™ device.

(a) is a cutaway perspective view showing a marker of the present invention compressed inside of a cannula of a side hole cannula delivery system using a biopsy needle.

FIG. 7(b) is a cross-sectional view of the delivery system of FIG. 7(a) showing the cannula with grips and a plunger for placing the marker in a patient.

FIGS. 8(a) and 8(b) shows a marker according to the present invention placed in compression and being delivered through a needle.

FIGS. 9(a) to 9(d) show a “loop” marker according to the present invention suitable for delivery by an expandable balloon.

### DETAILED DESCRIPTION OF THE INVENTION

The subject invention is comprised of a series of implantable sterile and biologically inert metal devices designed to mark the location of the site of a lesion in tissue. These markers are designed to optimize their properties for subsequent metal detection. Such optimization of detection characteristics will be accomplished by creating a device, which, unlike all other existing localization devices, has been specifically designed to provide the maximum metal detection signal and the clearest directional information possible, given the necessities of small size, tissue stability, patient comfort and biocompatibility.

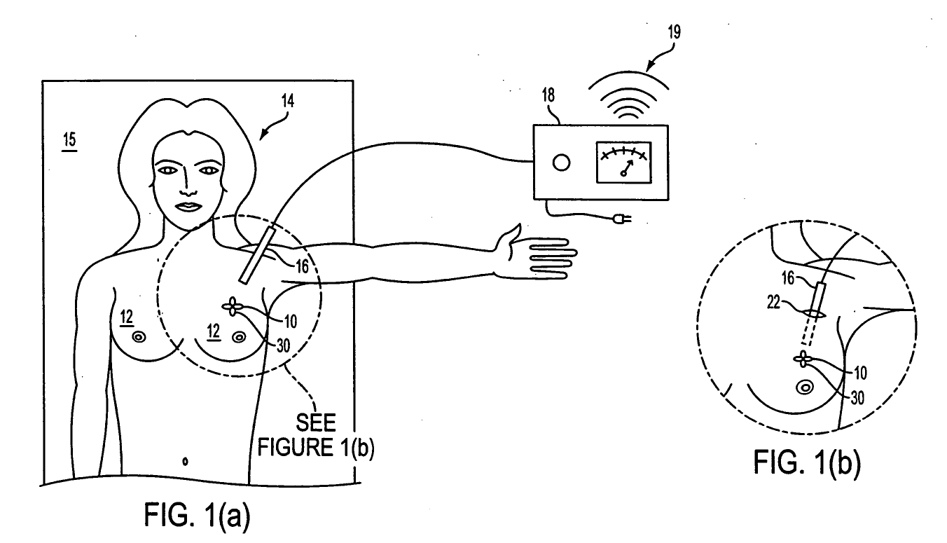

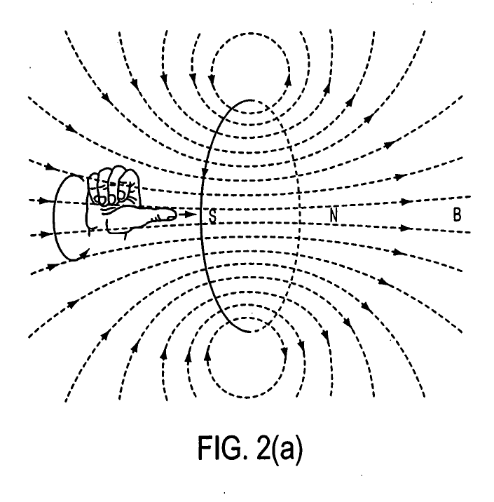

Turning now to the drawings, FIG. 1(a) shows a marker 10 according to the present invention implanted in a patient's breast 12 with the patient 14 positioned for surgery on a table 15. A metal detection probe 16 is illustrated in communication with a metal detector display 18 with audible output 19. A suitable metal detector and probe is illustrated in Trobec, R. et al., Metal Implant Localizers: Frontiers and Diagnostic Feasibility, Journal of Medical Engineering & Technology, 1996 (May/June: 20(3):134-140).

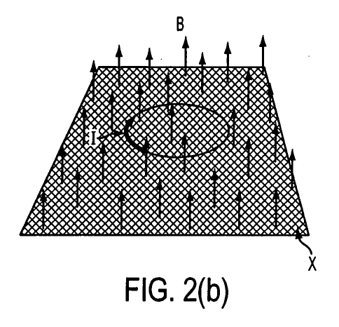

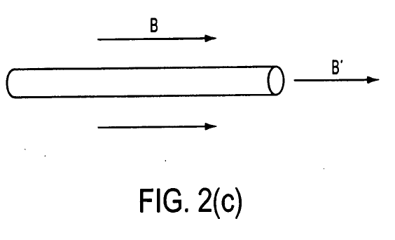

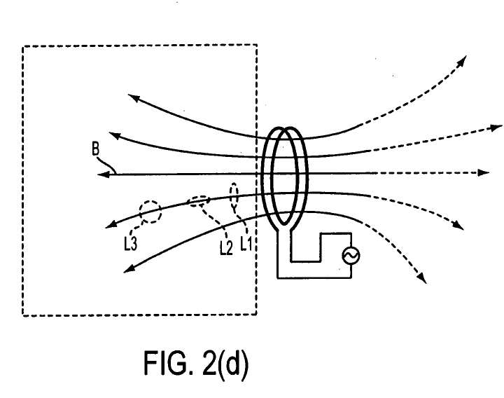

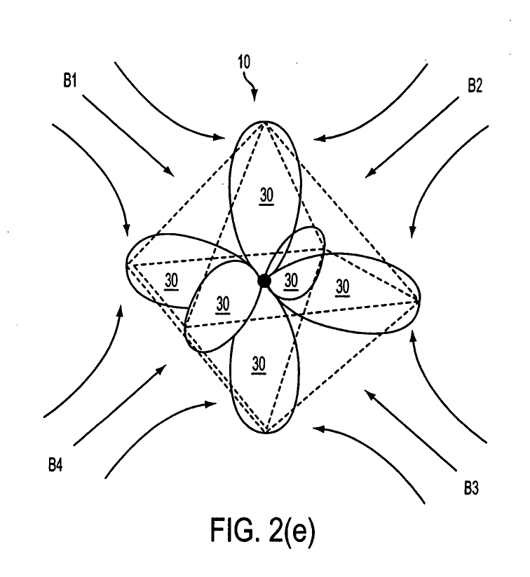

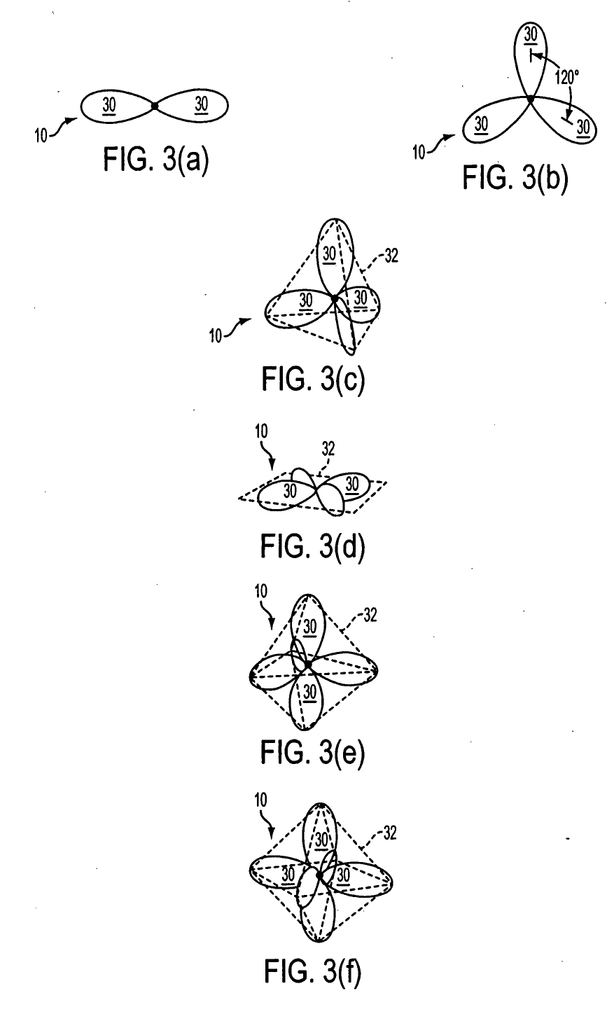

FIG. 1(b) illustrates a metal detection probe 16 inside a surgical dissection through incision 22 in patient 14 approaching a marker 10. The marker 10 has the physical characteristic to function as an antenna to the probe 16 by having at least one closed loop 30 that may be advantageously positioned for detection in a magnetic field of a metal detector, as described in FIGS. 2(a) to 2(e), below. An audio signal 19, which will vary in character (e.g., volume, pitch or intensity) depending upon the orientation of the probe 16 and its distance from the marker 10, provides direction to a site in a patient 14 to a surgeon or medical professional, i.e., the probe 16 will provide a differing output 18, 19 when the probe 16 is directed toward the site where the marker 10 has been placed than when it is oriented away from the marker 10. The term “site,” as used herein is intended to describe a site of a primary biopsy; a cavity site where from which a tissue sample has been removed, or any patient site where a marker is suggested by a medical practitioner.

By way of example, audio and other output 18, 19 may be enhanced or presented by use of a computer and/or a display screen to allow displays of probe direction and guidance.

Merely by way of explanation, and while not wishing to be bound by any theory of operation, it is believed the theory of the detection of the present invention may be illustrated as follows. FIGS. 2(a) to 2(e) show the believed theory of operation of the present invention, with FIG. 2(a) showing the shape and direction of a magnetic field (“B”) created by current flowing in loop (as in a metal detector) with the right hand rule of current flow illustrated in regard to the north (“N”) and south (“S”) pole. FIG. 2(b) shows the induction of a current (“I”) in a closed conducting loop by an increasing magnetic field B. As illustrated in FIG. 2(b), this effect is maximized when the closed loop lies substantially within a plane (“X”) that is orthogonal to the field lines of B. FIG. 2(c) shows the magnetization B′ of a ferromagnetic object in an applied external magnetic field B. As illustrated, this effect is maximized when the orientation of the long axis of the object is substantially parallel to the magnetic field. Turning now to FIG. 2(d), and by way of illustration, the combined effects of current induction and magnetization of wire loops in an alternating magnetic field B, such as in a metal detector beam, are maximized when a marker of the present invention made of a ferromagnetic conductor has a plurality of loops such that the plane of at least one loop L1 is orthogonal to the magnetic field created by metal detector and at least one loop L2 or L3 is parallel to the magnetic field created by the metal detector.

Merely by way of further explanation, the inventor has discovered that for all metals, the detectability of the medical device that is due to the induction of current within the medical device is maximized when the medical device includes 1) a closed metallic loop, and 2) the orientation of this loop is orthogonal to the direction of the metal detection beam, i.e., the magnetic field generated by the metal detector. Thus, in certain embodiments of the present invention the inventive marker utilizes the advantageous properties for metal detection of an appropriately designed “loop” antenna. Metal detectors often rely on the induction of current within the target and a shape that readily allows the current to flow in a loop facilitates such induction. Although any piece of metal large enough may be detectable by a metal detector due to the induction of eddy currents on the surface of the metal object, the use of a more efficient antenna design will allow the device to be constructed of the least amount of material possible, thus simplifying delivery and improving patient tolerance.

Further, metals differ in those physical characteristics that may produce detectability by a metal detector. Specifically, metal detectors may more readily detect ferromagnetic metals than paramagnetic metals due to their unique magnetic properties. In addition to undergoing current induction, ferromagnetic metals also interact with the beam of a metal detector by undergoing recurrent magnetization and demagnetization in the changing magnetic field and are thus additionally detectable due to the power loss associated with magnetic hysteresis. See Trobec, R. et al., Metal Implant Localizers: Frontiers and Diagnostic Feasibility, Journal of Medical Engineering & Technology, 1996 (May/June: 20(3): 134-140). The inventor has discovered that for ferromagnetic metals the additional detectability of the medical device due to the power loss associated with the magnetic hysteresis cycle within the medical device is maximized when the medical device includes 1) a linear element, and 2) the orientation of this linear element is parallel to the direction of the metal detection beam, i.e., the magnetic field generated by the metal detector. Accordingly, a medical device made of a ferromagnetic metal that has a plurality of closed metal loops such that 1) not only is at least one loop substantially orthogonal to the metal detection beam for a given angle of the beam, but in addition 2) at least one set of linear elements comprising another loop is substantially parallel to the metal detection beam for a given angle of the beam, will demonstrate an advantageous increase in its detectability by a metal detector.

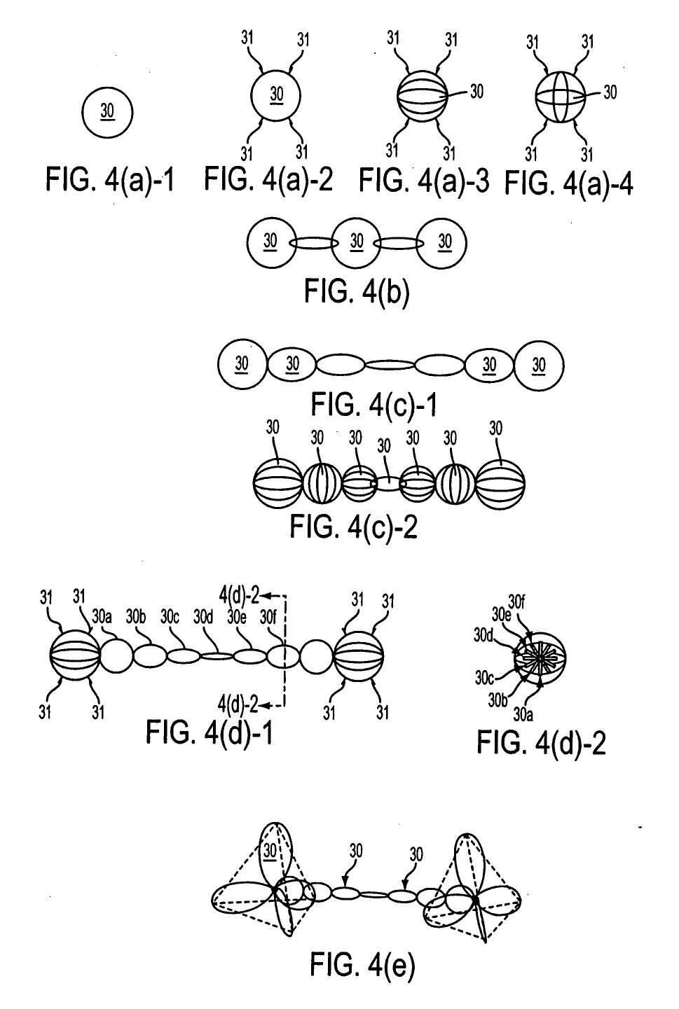

In practicing the present invention, the practitioner will apply a metal detection beam from various angles in an attempt to locate the inventive marker or medical device and ascertain its depth within the tissue. By incorporating a unique series of multiple interlocking metal loops of various orientations, such at those illustrated in FIGS. 3(a) to 5(e), the likelihood that a detection beam may encounter an orthogonal plane described by one of these interlocking loops as well as a parallel set of linear elements is advantageously increased. As shown in many of the markers in FIGS. 3(a) to 5(e), the loops are positioned at various angles to one another such that a metal detection beam will favorably impact at least one of the loops when the marker is deployed into the patient's tissue for the purpose of subsequent removal guided by metal detection.

These principles are illustrated by way of example in FIG. 2(e) in the design of a marker 10 according to the present invention, where the structure is such that notwithstanding the orientation of the marker 10 within the human body, at least one loop 30 is orthogonal to a magnetic field from the direction of B1, B2, B3, B4, or other directions not pictured. Accordingly, the marker 10 has a plurality of closed metal loops such that at least one loop is substantially orthogonal to the metal detection beam and one loop is substantially parallel to the metal detection beam for a given angle of approach. This design allows the inventive devices to be readily detectable by metal detectors while still using similar materials for fabrication and remaining within the range of sizes commonly employed by other marking devices that do not use metal detection for guidance.

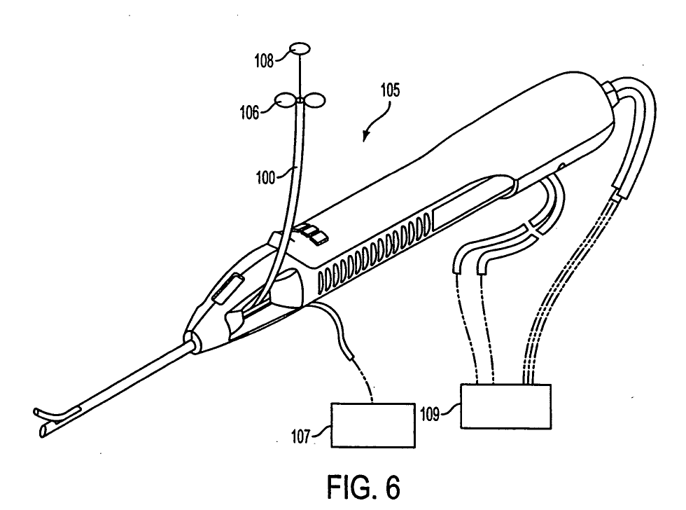

Within these guiding principles, the inventive marker may be formed into a number of desired shapes to achieve maximum detection. By way of further illustration, marker shapes may be patterned from (i) electron orbits of FIGS. 3(a) to 3(f); (ii) chain-like configurations of loops of FIGS. 4(a)-1 to 4(e); and (iii) various barbell shapes of FIGS. 5(a)-5(e) or combinations thereof.

Turning now to FIGS. 3(a) to 3(f), the markers 10 have shapes that are patterned from electron orbits. FIG. 3(a) shows a linear closed loop 30; FIG. 3(b) shows a planar closed loop 30; FIG. 3(c) shows a tetrahedral closed loop 30 with the dotted lines illustrating the shape 32; FIG. 3(d) shows square planar closed loop 30 with the dotted lines illustrating the shape 32; FIG. 3(e) shows a trigonal bipyramidal closed loop 30 with the dotted lines illustrating the shape 32; and FIG. 3(f) shows a octahedral closed loop 30 with the dotted lines illustrating the shape 32.

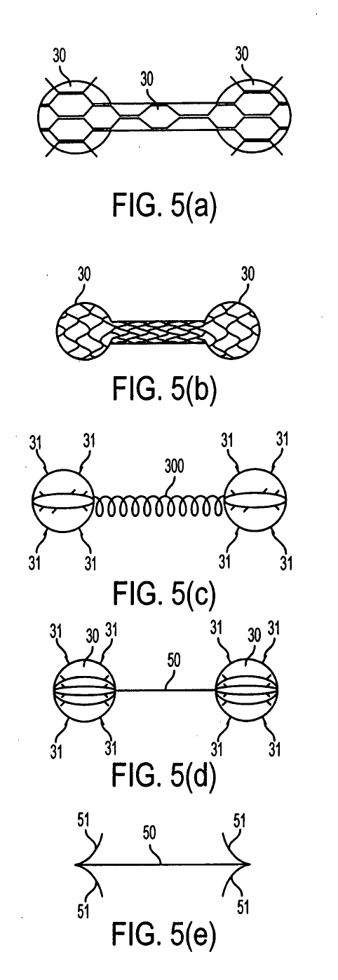

Turning now to FIGS. 4(a)-1 to 4(e), the marker of present invention may have various shapes patterned from chain-like configurations of loops. FIG. 4(a)-1 shows a single element loop 30. FIG. 4(a)-2 shows a single element loop 30 having anchors 31. FIGS. 4(a)-3 and 4(a)-4 show possible combinations of the loops of FIG. 4(a)-1 and 4(a)-2. FIG. 4(b) shows a chain that allows the loops 30 to move freely. FIG. 4(c)-1 and 4(c)-2 each show a chain with each loop 30 rotated with respect to the next loop. FIG. 4(d)-1 shows single a chain of loops 30 each rotated with respect to each other, as shown in the end view of FIG. 4(d)-2 showing loops 30(a)-(f), which are anchored by the spherical loops of FIG. 4(a). FIG. 4(e) shows a chain of loops rotated with respect to each other and anchored by the tetrahededral shape of FIG. 3(c).

Turning to FIGS. 5(a) to 5(e), markers of the present invention may have shapes patterned from barbell shapes. FIG. 5(a) shows expandable polygonal cells of welded wire having loops 30. FIG. 5(b) shows an expandable braided wire mesh having loops 30. FIG. 5(c) shows a spring coil 300 having loops anchoring its ends. FIG. 5(d) shows a central stiff segment 50 anchored by the spherical loops of FIG. 4(a)-3 having loops 30 and anchoring feet 31 to prevent migration. FIG. 5(e) shows a central stiff segment 50 for surgical purposes with barbs 51 to prevent migration.

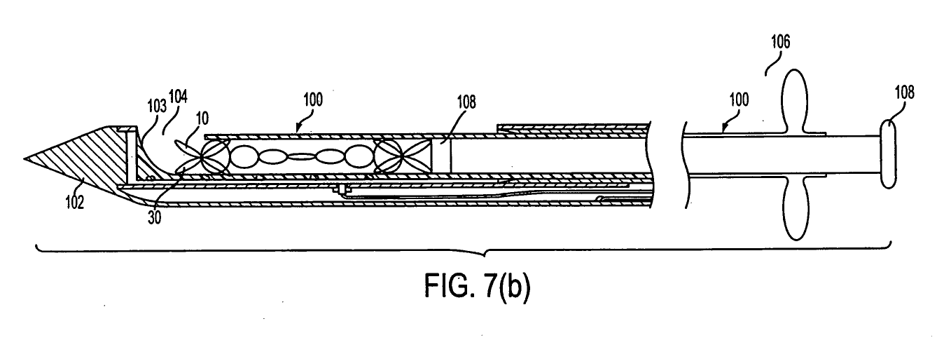

The inventive medical device marker 10 is fabricated by wire or etched components assembled by laser welding or the like to form a plurality of loops. As illustrated, the closed wire loops of FIGS. 3(a) to 5(e) can be readily compressed into a delivery system, as shown in FIGS. 7(a) to 9(d), and reliably expanded into various three dimensional orientations, either to be placed alone or anchor the ends of longer a longer “antenna.” When the inventive marker is positioned within the body, at least one of the loops is substantially orthogonal to the metal detection beam for a given directional source of the beam. Thus, the metal detector will give a maximal output 18, 19, from a given angle of approach of the beam, providing guidance or roadmap to locate the marker at the site of the lesion.

Another advantage of the present invention is that it can be used to identify a site within the body of a patient where a tissue sample 1) is to be taken or 2) has already been removed. Such a device should be capable of being detected by a metal detection magnetic field or beam of a metal detection device, as has been illustrated. Such devices may be implanted into any tissue within the body. The devices can be implanted into breast, lung, liver or other tissues at any convenient time prior to a surgical procedure. The devices remain stable at the site of implantation for the purposes of subsequent surgical removal assisted by a metal detection apparatus, as described below. Importantly, as described herein, it is believed that the length and diameter of an inventive marker having the above-identified shapes can provide a basis to maintain the position in tissue even when placed in tissue that is under compression and then decompression.

By way of illustration, certain metals are more appropriate than others for use in conjunction with particular imaging technologies. For example, the use of ferromagnetic metals may not be optimal in conjunction with MRI technology because such metals may 1) be more prone to attraction into the strong magnetic field used in this application and therefore present a risk of dislodgment from the delivery system or within the patient or 2) distort subsequent MRI images due to “magnetic susceptibility artifacts”. Thus, the invention envisions the use of either or both paramagnetic and ferromagnetic metals.

The inventive devices can be made of a metallic material such that the devices may be delivered to a desired location in the patient's body in compressed form and then expanded upon deployment. In certain embodiments, the metal compositions have shape memory characteristics to facilitate such expansion after deployment. Nickel titanium (also known as nitinol) is an exemplary shape memory alloy, and other metals may be used or applied to the invention; i.e., certain types of cobalt or stainless steel that also display such shape memory. Furthermore, the properties of any metal can be modified to a great extent by plating or joining with other metals, changes in alloy composition, mechanical working, heat treatment, etc, to advantageously combine or alter certain desired characteristics (i.e. magnetic or shape memory properties).

Specifically, optimization of those factors that may improve detectability by a metal detector while allowing for the shape memory characteristics desirable for the successful deployment of the invention is well within the purview of the skilled artisan. Alternatively, a metal with certain desired detection characteristics but without exemplary shape memory may be actively expanded from its compressed form by the application of an external force (i.e. balloon expansion or contraction with a wire) to accomplish the formation of the desired shape after positioning in the patient's body. Moreover, the various marker elements of FIGS. 3(a) to 5(e) can be “mixed and matched” as needed to optimize marker performance.

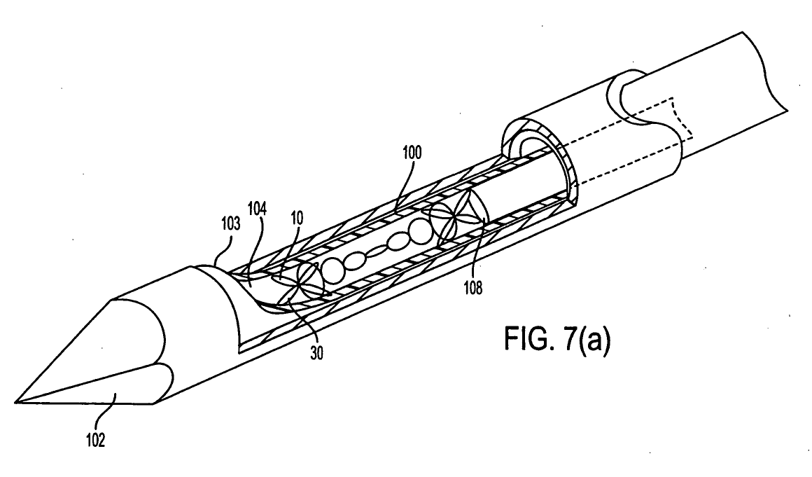

Turning now to FIGS. 6 to 9(d), the marker of the present invention may be delivered or placed into the body by several known techniques. By using a metal with optimal physical properties made into a thin wire, the marker or device may be tightly packed into a delivery system, as described below. After an accurately positioned needle has accessed a target using any conventional guidance method (mammography, stereotactic mammography, ultrasound, MRI, CT, etc.), the inventive device may be deployed. When released, the inventive device will be expanded, assuming the desired shape. As illustrated in FIG. 3(a) to 5(e), anchoring elements may anchor the device to prevent migration. The device will then remain in place until such time as its removal may be desired. Delivery systems adapted for use with multiple differing needle types and biopsy instruments will be easily designed to match the diameter and geometry of distal opening for each specific application.

The inventive medical devices are detectable by a metal detection beam of a metal detection device. The inventive devices can be detectable by any of the several methods of metal detection known. Since a particular form of detector may have certain strengths or weaknesses in a specific application (such as its coil design, oscillation frequency, power output, detection sensitivity, depth of detection, directionality of detection, ability to detect specific metal types, size, shape, controls, display, etc) the present invention seeks to utilize a construction which, by nature of its shape and metallic composition, will maximize detectability regardless of the detection device employed.

In one embodiment of this aspect of the present invention, the inventive medical devices may work to maximum advantage using the several types of detectors available or under development in combination. A multi-stage detection procedure can utilize multiple specialized probes in sequence. Detection by ultrahigh sensitivity detectors prior to the commencement of the surgical procedure will establish the general location of the target before the incision is made. Next, continuous intraoperative monitoring by means of small sterile probes inside the dissection will allow any surgical approach to be used and adjusted as the target is approached. This will provide for surgical precision and flexibility of surgical approach.

Several different implantable devices according to the present invention are possible, each tailored for use in specific clinical circumstances. Lesions of differing diameters require inventive markers of different length or radii, lesions localized in compression require differing anchoring systems (i.e. to provide correction for the “accordion effect” responsible for z-axis errors), large core biopsy cavities require larger diameter devices or coating with bioabsorbant materials, markers to be detected through thicker amounts of tissue require more mass, differing guidance techniques (e.g. MRI, X-Ray) require construction by specially compatible materials, etc. Similarly, as detector technology evolves, differing antenna shapes or sizes will provide characteristics favorable for certain detectors.

By way of further illustration, a breast in compression will have a needle inserted along an axis of compression (“z-axis”). Although the position of the lesion may be localized while the breast is in compression, the reexpansion of the breast may change the relationship of the lesion to the marker due to the “accordion effect”. This is one reason a marker may benefit from having length along the z-axis as well as proximal and distal anchors. Furthermore, such a longer device will conform better to the cylindrical shape of the cavity created by a series of large core biopsies and thus better fill the space left following the completion of such a biopsy.

By way of further example, FIG. 6 shows a flexible cannula 100 positioned within a conventional Mammotome™ device 105, available from Ethicon Endosurgery of Johnson & Johnson, which has a plunger 108 and grips 106 to position an inventive marker (not shown). The Mammotome™ device 105 includes an energy unit 107 and a control module 109. The use of a flexible cannula 100 allows the delivery of an inventive marker through other like devices well known in the art.

FIGS. 7(a) and 7(b) show an inventive marker 10 compressed inside of a cannula 100 positioned within a biopsy needle 102, such as a needle of a conventional Mammotome™ device shown in FIG. 6. The cannula 100 uses a side hole 104 to exit from the biopsy needle 102. A deflecting ramp 103 positioned at the end of the cannula 100 provides the direction for the marker 10 to exit from the cannula 100. The marker 10 is compressed inside of the cannula 100 and is positioned by the conventional application by the practitioner of the grips 106 and plunger 108; that is, plunger 108 pushes marker 10 out of cannula 100 through side hole 104. Upon exiting the cannula 100, the marker 10 expands and partially or fully decompresses. Upon decompression, the marker 10 will display a plurality of loops 30, one or more of which will be positioned orthogonal to the metal detector field. As described above, it is believed that the length of the marker 10 will also provide for precise location of the marker 10, particularly when positioned within breast tissue.

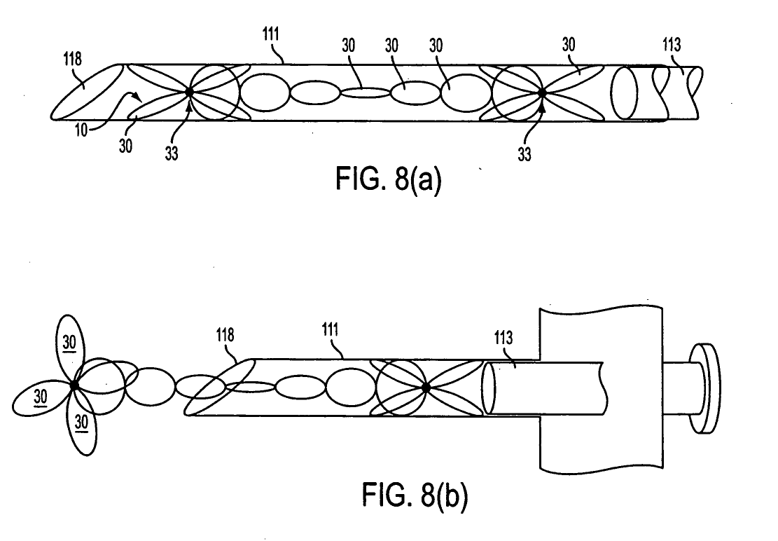

FIGS. 8(a) and 8(b) show an inventive marker 10 having a chain and anchoring loops in compression inside of a needle 111 for delivery to the desired portion of the body. As illustrated, the marker 10 is made of a chain of loops 30 anchored by tetrahedral loops 33 compressed inside of needle 111. As a plunger 113 moves forward in response to applied pressure by the practitioner, the marker 10 is moved through the distal end 118 of the needle 111. Upon exiting from the distal end 118 of the needle 111, the marker 10 expands by exercise of the shape memory of the marker 10.

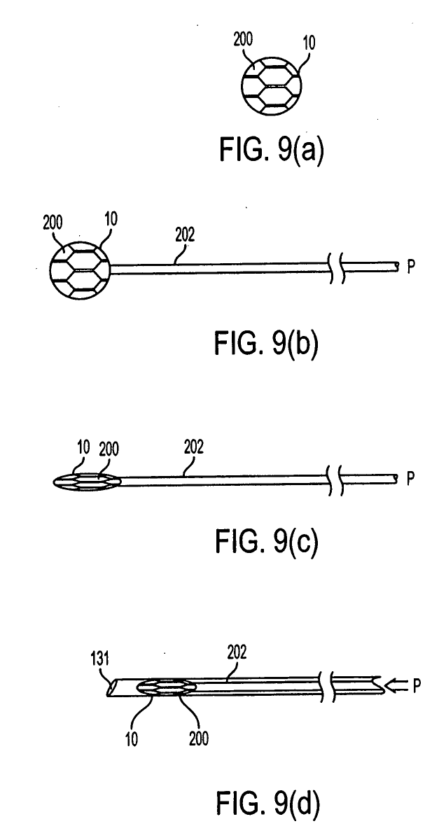

FIGS. 9(a) to 9(d) show a “loop” marker 10 according to the present invention suitable for delivery to a patient by an expandable balloon 200 with a needle 131. FIG. 9(a) shows an expanded marker 10, which can alternatively be a marker as illustrated in FIGS. 4(a)-1 to 4(a)-4. Balloon 200 has a source of pressure “P” to for expansion; FIG. 9(b) shows a balloon tip catheter 202 that fits inside the marker 10 with the marker 10 shown expanded. FIG. 9(c) shows marker 10 compressed around deflated balloon 200. FIG. 9(d) shows marker 10 positioned around balloon 200 and within needle 131 such that when marker 10 is moved beyond the distal end of the needle 131, pressure may be applied to inflate balloon 200 and expand marker 10.

Based upon the foregoing description, the applications of the invention are many and varied. By way of example, the importance of accurate needle localization prior to breast biopsy or excision of cancer has been well described. Needle localization is generally accomplished using mammography a guide. The use of stereotactic needle localization has been described, but carries with it certain significant limitations. These limitations are predominantly due to the fact that, although accurate localization within millimeters of the target is readily accomplished when the breast is compressed within the stereotactic apparatus, upon release of compression significant errors in the depth of needle placement along the “z axis” are frequently encountered. This limitation has been understood as the so called “accordion” effect and is due to the re-expansion of the breast after the release of compression and may result in difficulty maintaining precise localization of a marker along the z-axis after the release of compression.

In one embodiment of the present invention this limitation is overcome by employing a marker which can be deployed with a portion of its length consistently maintained alongside the target after the release of compression following stereotactic needle localization.

Standard hookwires can be deployed with barbed or hooked ends at the distal aspect of a wire, to prevent only proximal migration of the wire. By the addition of proximal anchoring elements oriented in the opposite direction to also limit distal movement of the inventive device, the accordion effect could be eliminated or significantly reduced. Thus, the presence of looped anchoring elements at the distal as well as the proximal aspect of the central segment of the inventive marker as illustrated by way of example in FIGS. 4(a)-1 to 5(e) will prevent both forward and reverse migration of the central segment with respect to the target, therefore effecting accurate localization which is maintained when the breast is released from compression. A different length of central segment can be employed depending on the thickness of the breast in compression as well as the actual geometry of the target.

Also, a known charcoal marking technique can be used to additionally mark the position of the inventive metallic marker, such as described by D. Mullen, supra.

With such a device according to the present invention needle localization could be easily accomplished using stereo mammography equipment. An advantage of the inventive device is that it significantly increases the range and number of procedures that can be performed on stereotactic mammography equipment. The stereotactic mammography equipment itself, as well as the rooms in which this equipment is deployed, represents a significant capital expense for a facility offering such procedures. Allowing accurate localizations to be performed on such equipment enhances the productivity of these rooms.

Furthermore, patients will experience significant benefits in the use of the inventive procedure. It is well known that the prone position offered by stereotactic mammography improves patients' tolerance of needle procedures in the breast by decreasing the incidence of vasovagal response as well as the perception of pain. Procedure time is significantly decreased by the use of digital receptors, and precision is improved by the stereotactic technique.

Although the designs described above are favored for their simplicity, economy of material and ease of insertion, many other similar designs may be envisioned. In fact, any expandable cage constructed of an appropriate metal that may be expanded passively (i.e. by metal memory) or actively (i.e. by balloon inflation) into virtually any polyhedral, elliptical or spheroid shape will create a surface which may have similar properties for metal detection as the present design. Not withstanding such modifications in size, shape, material or method of insertion, and recognizing that other metal devices currently in use may be sub-optimally detectable by metal detectors, no other marker has been specifically designed and proven to be optimized for metal detection. Thus, the present invention is meant to cover as broadly as possible the use of any implanted metal device to serve as an antenna for subsequent localization by metal detection.

There is described herein only a few embodiments of the present invention and but a few examples of its versatility. It is to be understood that the invention is capable of use in various other combinations and environments and is capable of changes or modifications within the scope of the inventive concept as expressed herein. Thus, for example, those skilled in the art will recognize, or be able to ascertain, using no more than routine experimentation, numerous equivalents to the specific devices and procedures described herein. Such equivalents are considered to be within the scope of this invention.

What is claimed is:

1. A medical marker for marking a site within the body of a mammalian patient which is capable of being detected by a metal detection beam of a metal detection device, the marker comprising at least one loop positionable within the patient to be substantially orthogonal to said metal detection beam for a given directional source of the beam; wherein the entirety of the marker is implantable within the patient's body.

2. The marker of claim 1, wherein the metal is selected from the group consisting of a memory metal or ferromagnetic metal or combination thereof.

3. The marker of claim 2 wherein the memory metal is nitinol.

4. The marker of claim 1 wherein the marker is collapsible such that the marker expands when it is delivered to the site of the tissue to be resected.

5. The marker of claim 1 wherein the marker is selected from the group consisting of shapes patterned from electron orbits, chains of loops or barbell shapes.

6. The marker of claim 1, wherein said marker has at least one loop positionable within a patient to be maintained substantially parallel to said metal detection beam.

7. The marker of claim 1 wherein the marker comprises a ferromagnetic material.

8. The marker of claim 1 wherein said marker is positioned within a needle or a cannula for delivery and placement at the site within the patient.

9. A medical device for marking a site within the body of a mammalian patient, comprising:
a) a needle having a proximal end and a distal end configured to pierce tissue; and
b) a marker disposed within said lumen for transport through said needle; said marker compressible within said needle and detectable by a metal detection beam of a metal detection device when expanded; said marker comprising least one loop of a plurality of loops positionable substantially orthogonal to said metal detection beam for a given directional source of the beam, wherein the entirety of the marker is implantable within the patient's body.

10. The device of claim 9, wherein at least one loop of said marker loops is positionable in the patient substantially parallel to said metal detection beam for a given directional source of the beam.

11. A medical device for marking a site within the body of a mammalian patient comprising:
a) a needle having a proximal end and a distal end, configured to pierce tissue of a patient; and
b) an expandable marker disposed around a balloon within said needle for transport within the needle, said marker detectable by a metal detection beam of a metal detection device, said marker comprising least one loop of a plurality of loops positionable in the patient substantially orthogonal to said metal detection beam for a given directional source of the beam, wherein the entirety of the marker is implantable within the patient's body.

12. The device of claim 11, wherein at least one loop of said marker loops is positionable in the patient substantially parallel to said metal detection beam for a given directional source of the beam.

13. A system for detecting a site within the body of a mammalian patient comprising:
a) a marker capable of being detected by a metal detection beam of a metal detection device, the marker comprising a plurality of loops wherein at least one of said loops is substantially orthogonal to said metal detection beam for a given directional source of the beam, wherein the entirety of the marker is implantable within the patient's body; and
b) a metal detection device having a signal that provides guidance or direction to the location of a marker in patient.

14. The system of claim 13, wherein at least one loop of said marker loops is positionable in the patient substantially parallel to said metal detection beam for a given directional source of the beam.

15. A method for diagnosis and treating a patient comprising:
a) removing a portion of a putative lesion at a biopsy site;
b) marking a location at said biopsy site by inserting into the biopsy site a marker capable of being detected by a metal detection beam of a metal detection device, the marker comprising a plurality of loops wherein at least one of said loops is substantially orthogonal to said metal detection beam for a given directional source of the beam, and wherein the entirety of the marker is implanted within the patient's body;
c) detecting the location of the marker within said biopsy site using said metal detection device; and
d) removing the tissue proximate to said marker.

16. The method of claim 15, wherein at least one loop of said marker loops is positionable in the patient substantially parallel to said metal detection beam for a given directional source of the beam.

17. A method for marking a selected area of tissue comprising the steps of:
a) inserting a cannula through a previously positioned large core biopsy device or inserting a needle into a large core tract formed by the large core needle during a biopsy, and
b) placing a marker detectable by a metal detector within a selected area of tissue in a biopsy site via the cannula or the large core tract, said marker having at least one loop positionable to be orthogonal to a metal detection beam of a metal detection device for a given directional source of the beam, wherein the entirety of the marker is implanted within the patient's body.

18. The method of claim 17, wherein said marker has at least one loop positionable in the patient substantially parallel to said metal detection beam for a given directional source of the beam.

19. The method of claim 17 wherein the marker is selected from the group consisting of shapes patterned from electron orbits, chains of loops or barbell shapes.

20. The method of claim 17 wherein said marker is selected from the group consisting of a memory metal, ferromagnetic material or combination thereof.

21. A method for minimizing the need for repeat localizations following breast biopsy comprising the steps of:
a) performing a large core needle biopsy in a breast; and
b) utilizing a large core biopsy tract formed during a large core needle biopsy as a conduit for injection of a marker detectable by a metal detector, said marker having at least one loop positioned in the breast orthogonal to a magnetic field generated by a metal detector for a directional source of the beam, said marker positioned into a biopsy cavity within the large core tract, wherein the entirety of the marker is implanted within the patient's body.

22. The method of claim 21, wherein said marker has at least one loop positionable in the patient substantially parallel to said metal detection beam for a given directional source of the beam.

23. A kit for marking a site within the body of a mammalian patient which is capable of being detected by a metal detection beam of a metal detection device comprising:
a) a marker having least one loop being positionable within the patient to be maintained substantially orthogonal to said metal detection beam for a given directional source of the beam, wherein the entirety of the marker is implantable within the patient's body;
b) a metal detector suitable to detect the location of said marker when said marker is positioned within the patient; and
c) a delivery device suitable to convey the marker into the patient's tissue and place the marker in the patient's tissue.

24. The kit of claim 23, wherein the marker has at least one loop positionable in the patient substantially parallel to said metal detection beam for a given directional source of the beam.

25. The kit of claim 23, wherein said metal detector includes an audio signal.

26. The kit of claim 23, wherein the kit includes a computer and/or a display screen.

27. The kit of claim 23, wherein a portion of said marker has a substantially spherical or ovoid shape.

28. A marker for marking a site within the body of a mammalian patient which is capable of being detected by a metal detection beam of a metal detection device comprising at least one segment of the marker positionable within the patient to be substantially parallel to said metal detection beam for a given directional source of the beam; wherein the entirety of the marker is implantable within the patient's body.

29. The marker of claim 28 wherein the marker is selected from the group consisting of shapes patterned from electron orbits, chains of loops or barbell shapes.

30. A medical device for marking a site within the body of a mammalian patient comprising:
a) a cannula having a proximal end and a distal end and defining a lumen therebetween; and
b) a marker disposed within said lumen for transport through said cannula; said marker detectable by a metal detection beam of a metal detection device, said marker comprising least one loop of a plurality of loops positionable in the patient substantially orthogonal to said metal detection beam for a given directional source of the beam, wherein the entirety of the marker is implantable within the patient's body.

31. The device of claim 30, wherein at least one loop of said marker loops is positionable in the patient substantially parallel to said metal detection beam for a given directional source of the beam.

32. The medical device of claim 30 further comprising a charcoal-containing solution positioned with said cannula wherein an effective amount of said solution is released by the cannula when a distal end is at said site.

33. A medical marker for marking a site within the body of a mammalian patient which is capable of being detected by a metal detection beam of a metal detection device comprising:
a) at least one loop being positionable within the patient to be substantially orthogonal to said metal detection beam for a given directional source of the beam; and
b) at least one linear element being positionable within the patient to be substantially parallel to said metal detection beam for a given directional source of the beam; and
c) a substantially spherical or ovoid shaped portion; wherein the entirety of the marker is implantable within the patient's body.

34. The marker of claim 1, wherein the marker has a substantially spherical or ovoid shaped portion.

35. The marker of claim 28, further compromising a second portion of a substantially spherical or ovoid shape supporting said first one segment.
